(19) United States
(12) Patent Application Publication
(10) Pub. No.: US 2025/0170648 A1
(43) Pub. Date: May 29, 2025

(54) SELECTIVE COLORING BY MEANS OF COLORING SOLUTIONS IN 3D PRINTING

(71) Applicant: Ivoclar Vivadent AG, Schaan (LI)

(72) Inventors: Hendrik JOHN, Buchs (CH); Frank ROTHBRUST, Röns (AT); Sebastian KROLIKOWSKI, Richterswil (CH); Christian RITZBERGER, Grabs (CH)

(21) Appl. No.: 18/952,241

(22) Filed: Nov. 19, 2024

(30) Foreign Application Priority Data
Nov. 29, 2023 (EP) 23213096.3

(51) Int. Cl. B22F 10/14; B22F 3/10; B22F 10/50; B22F 12/00; B22F 12/55; B33Y 10/00; B33Y 30/00; B33Y 80/00

(52) U.S. Cl. CPC B22F 10/14; B22F 3/10; B22F 10/50; B22F 12/38; B22F 12/55; B33Y 10/00; B33Y 30/00; B33Y 80/00

(57) ABSTRACT

A method of producing a dental restoration (100) by jet printing, including the steps of jet printing (S101) one or more layers of the dental restoration using a ceramic slurry; and jet printing (S102) a coloring solution onto the one or more layers.

### CROSS-REFERENCE TO RELATED APPLICATIONS

This application claims priority to European Patent Application No. 23213096.3 filed on Nov. 29, 2023, which is incorporated herein by reference in its entirety.

### TECHNICAL FIELD

The present invention relates to a method of producing a dental restoration by jet printing and to a production apparatus for producing a dental restoration by jet printing.

### BACKGROUND

Differently colored, solvent-based ceramic slurries, such as those made from zirconium dioxide, can be selectively applied drop by drop in a large number of layers using a jet process (inkjet process). However, processing highly filled ceramic slurries places high demands on the print head used in terms of particle size, filling level, viscosity and abrasion. Jet printing (jetting) of aqueous ceramic slurries is an alternative to solvent-based slurries. In this, the jetted material layer is dried without cracking.

US 20200017699, 20120308837 and 20100249305 are directed to 3D printing and are hereby incorporated by reference in their entirety.

### Summary

It is the technical task of the present invention to improve a multi-color 3D printing process using an inkjet for ceramic slurries.

This task is solved by subject-matter according to the independent claims. Technically advantageous embodiments are the subject-matter of the dependent claims, the description and the drawings.

According to a first aspect, the technical task is solved by a method of producing a dental restoration by jet printing, comprising the steps of jet printing one or more layers of the dental restoration by means of a ceramic slurry; and jet printing a coloring solution onto the one or more layers. This allows the selective application process of the slurry and a selective coloring process to be separated from each other. The same or different print heads can be used for the application process and the coloring process.

If, on the other hand, pre-colored slurries are used, which also have different translucencies and/or different mechanical properties, each of these slurries is assigned its own print head. In contrast, the method can reduce the number of print heads for applying the slurries, as the coloring is carried out separately from the slurry build-up by subsequently jet printing coloring solutions.

In a technically advantageous embodiment of the method, the one or more layers are dried before jet-printing the coloring solution. This achieves the technical advantage, for example, that the coloring solution diffuses less and the local homogeneity of the coloring is improved.

In a further technically advantageous embodiment of the method, the applied coloring solution is fixed by means of an alkaline solution. The alkaline solution can be applied to the layer after or before the coloring solution is applied. This achieves the technical advantage, for example, of preventing the coloring solution from running.

In a further technically advantageous embodiment of the method, the ceramic slurry has a basic pH. This achieves the technical advantage, for example, that the coloring solution is automatically fixed by the slurry.

In a further technically advantageous embodiment of the method, the coloring solution is jet-printed onto the one or more wet layers. This achieves the technical advantage, for example, that a precipitation reaction of the coloring solution occurs and the method can be carried out more quickly.

In a further technically advantageous embodiment of the method, the coloring solution is fixed by contact with the ceramic slurry. This achieves the technical advantage, for example, that the method is carried out more efficiently.

In a further technically advantageous embodiment of the method, the one or more wet layers are dried together with the applied coloring solution. This achieves the technical advantage, for example, that the next layer can be applied immediately.

In a further technically advantageous embodiment of the method, the steps for producing the dental restoration are repeated. This achieves the technical advantage, for example, that the entire dental restoration can be built up and colored.

In a further technically advantageous embodiment of the method, the dental restoration is sintered in a sintering furnace. This achieves the technical advantage, for example, that a dental restoration with high strength can be produced.

In a further technically advantageous embodiment of the method, the produced dental restoration is subjected to a drying and/or debinding step prior to a sintering process. This drying and/or debinding step can take place in a separate thermal process or as an upstream process step in the sintering process. Drying is usually carried out at temperatures of 25° C. to 200° C., preferably 30° C. to 180° C. and particularly preferably 40° C. to 150° C. In addition, the humidity can be regulated between 10 and 90%, preferably 15 to 85% and particularly preferably 20 to 80%. Debinding is usually carried out at temperatures of 50° C. to 600° C., preferably between 100° C. and 600° C. and particularly preferably at 200° C. to 600° C. The heating rates are between 0.1 and 10K/min, preferably between 0.2 and 10K/min and particularly preferably between 0.5 and 10K/min.

According to a second aspect, the technical task is solved by a production apparatus for producing a dental restoration by jet printing, comprising a first print head for jet printing one or more layers of the dental restoration by means of a ceramic slurry; and a second print head for jet printing a coloring solution onto the one or more layers. The production apparatus achieves the same technical advantages as the method according to the first aspect.

In a technically advantageous embodiment of the production apparatus, the first print head and the second print head are integrated in a common print module. This achieves the technical advantage, for example, that the design of the production apparatus is simplified.

In a further technically advantageous embodiment of the production apparatus, the first print head and the second print head are independently controllable. This achieves the technical advantage, for example, that the slurry and the coloring solution can be applied independently of one another.

In a further technically advantageous embodiment of the production apparatus, the production apparatus comprises a dithering module for calculating intermediate color values by mixing at least two coloring solutions. This achieves the technical advantage, for example, of further improving the lifelike appearance of the dental restoration.

In a further technically advantageous embodiment of the production apparatus, the dithering module is configured to use different two-dimensional dithering patterns in successive layers of the dental restoration. This achieves the technical advantage, for example, of preventing the formation of stripe or moiré patterns in the dental restoration.

According to a third aspect, the technical task is solved by a jet-printing system, comprising a production apparatus for producing a dental restoration by jet-printing; at least one ceramic slurry for jet printing one or more layers of the dental restoration; and a coloring solution for jet printing onto the one or more layers. The jet-printing system achieves the same technical advantages as the method according to the first aspect.

In a technically advantageous embodiment of the jet-printing system the at least one ceramic slurry and/or coloring solution is stored in a container. This achieves the technical advantage, for example, that the ceramic slurry and/or coloring solution can be stored reliably.

In a further technically advantageous embodiment of the jet-printing system the container is replaceable. This achieves the technical advantage, for example, that the solutions can be exchanged easily.

### BRIEF DESCRIPTION OF THE DRAWINGS

Exemplary embodiments of the invention are shown in the drawings and are described in more detail below, in which:

FIG. 1 shows an illustration of different areas of an incisor;

FIG. 2 shows a schematic illustration of a dental restoration;

FIG. 3 shows a schematic illustration of a production apparatus for producing a dental restoration;

FIG. 4 shows a schematic view of a method of producing a dental restoration by jet printing;

FIG. 5 shows a schematic view of a further method of producing a dental restoration by jet printing;

FIG. 6 shows a table with the compositions of different coloring solutions; and

FIG. 7 shows a block diagram of the method of producing a dental restoration by jet printing.

### DETAILED DESCRIPTION

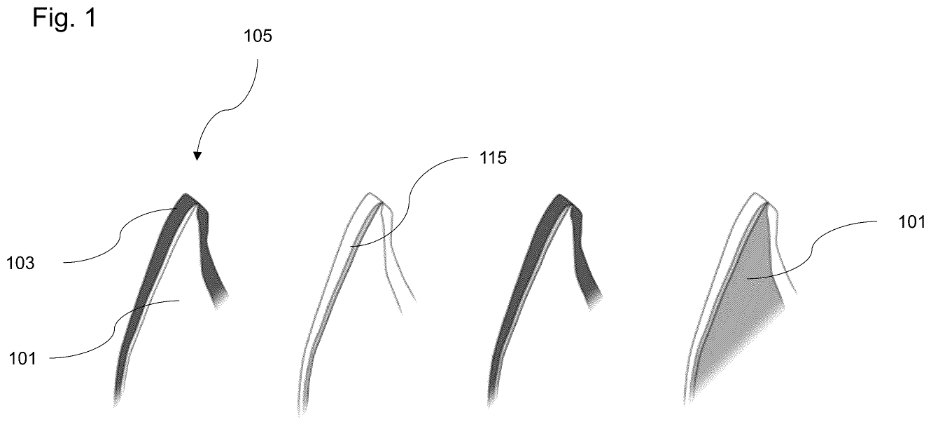

FIG. 1 shows an illustration of different areas of a tooth 105. The tooth 105 comprises an inner dentin core 101 and an outer tooth enamel 103. Between the dentin core 101 and the tooth enamel 103 there is optionally an intermediate area 115, which can also be assigned its own selection of ceramic slurries 109 and coloring solutions 107.

The opaque dentin core 101 is responsible for the basic coloring of the tooth 105. It shines through the tooth enamel 103 of the incisal edge. The tooth enamel 103 is naturally translucent. Translucency is the partial light transmittance of a body. In order to make a dental restoration look as lifelike as possible, this structure of the tooth 105 is also used in artificial dental restorations. For this purpose, materials with different optical properties are used in the production of the dental restoration.

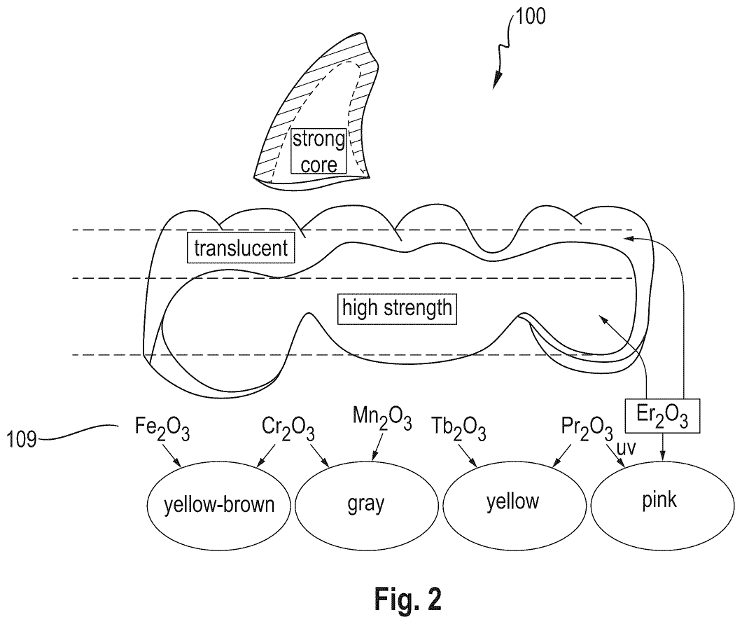

FIG. 2 shows a schematic illustration of a dental restoration 100. The dental restoration 100 serves as a dental prosthesis and is formed, for example, by a bridge, crown, veneer, inlay, onlay, abutment, full or partial prosthesis. The dental restoration 100 is built up using different ceramic slurries 109, for example. The ceramic slurry comprises partially stabilized ZrO₂ and/or Al₂O₃ particles. Al₂O₃ may already be incorporated in the same way as the stabilizing ions. Partial stabilization is usually achieved by incorporating Cao, MgO, Y₂O₃, La₂O₃ or CeO₂ and mixtures thereof in the ZrO₂. A preferred embodiment contains Y₂O₃ as a stabilizer in the quantity range of 1 to 10 mol %, in the particularly preferred quantity range of 2 to 8 mol % and in the more particularly preferred quantity range of 2.5 to 6 mol %. For this purpose, these slurries 109 are used selectively in the respective areas during the spatial production of the dental restoration 100 by means of a jet printing process.

The dental restoration 100 is built up by successive layers that are printed on top of each other. The ceramic powders of the slurries 109 can already be provided in predetermined translucencies. Mixing these slurries 109 results in the desired translucency of the dental restoration 100 in the respective spatial area.

After the selective material application of a layer of the slurry 109 using the jet printing process, this layer is dried without cracking by evaporating the water or solvent as a binder. What remains is a porous white body layer having a layer thickness of 1 um to 50 um and a density of at least 2.5 g/cm3. This process is repeated until the entire dental restoration 100 is built up spatially in layers.

In the case of ZrO₂ slurries 109, different yttrium-doped ceramic powders (3 mol % yttrium—3Y-TZP, 4 mol % yttrium—4Y-TZP, 5 mol % yttrium—5Y-TZP) can be used for the different strengths.

3Y-TZP=low translucency/high strength

4Y-TZP=medium translucency/medium strength

5Y-TZP=high translucency/low strength

However, if different pre-colored slurries 109 are used, all of which also having a different translucency and/or mechanical properties, a separate print head 111-1 is assigned to each of these slurries 109. For example, if a general four-color color scheme, such as CMKY (Cyan, Magenta, Yellow, Key/White), is used to cover the entire color space and slurries with different translucencies or strengths are also used in three variants, 3×4=12 slurries 109 are contained in 12 print heads. The production of these different slurries 109 is also complex. This large number of slurries 109 is kept and maintained as different articles.

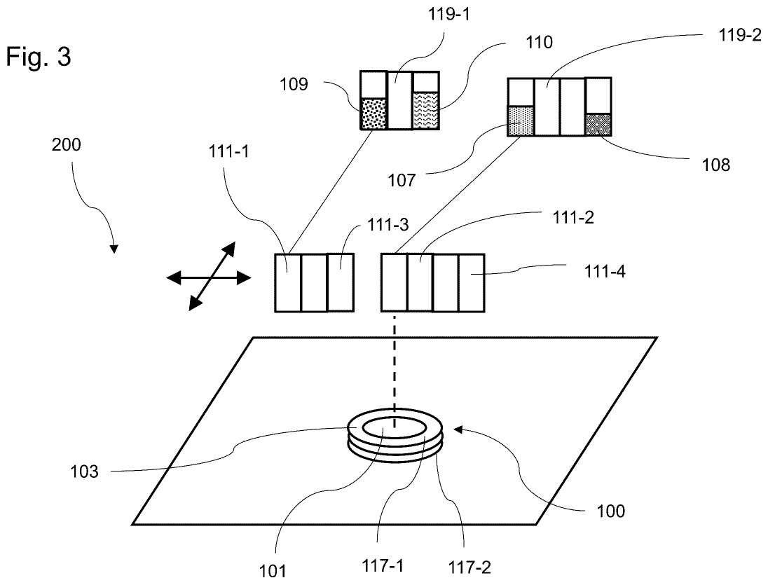

FIG. 3 shows a schematic illustration of a production apparatus 200 for producing a dental restoration 100 by jet printing (inkjet printing) of aqueous or solvent-based slurries 109. In order to additively manufacture ceramic, dental multi-material and multi-color restorations 100 by means of jet printing, base slurries 109 are processed.

The production apparatus 200 comprises a plurality of receiving containers 119-1, in which the base slurries 109 with different optical properties and the support material 110 are arranged. In addition, the production apparatus 200 comprises at least several receiving containers 119-2, in which the coloring solutions 107 and the alkaline solution 108 for fixing the coloring solution are received. The production apparatus for producing the dental restoration by jet-printing; at least one ceramic slurry; and the coloring solution for jet printing solution form together a jet-printing system.

The ceramic slurries 109 are each applied drop by drop in several layers 117-1, . . . , 117-n by means of an assigned print head 111-1 in order to build up the dental restoration 100 spatially in layers. The print head 111-1 is movable in two directions so that the slurry 109 can be printed at any position. Slurries 109 with a drop volume of typically 10 to 100 pL are used for selective material application, the use of which eliminates the time-consuming debinding process. Electrically controlled piezo elements, for example, are used to eject the droplets. Bubble jet technology can also be used to eject the slurry droplets. At least one further print head 111-3 is used for the application of the support material 110, through which the support material 110 can be selectively applied.

For the application of the coloring solutions 107, at least one further print head 111-2 is used, through which the coloring solutions 107 can be jet-printed onto one or more layers 117-1, . . . , 117-n. The print head 111-2 is also movable in two directions, so that the coloring solutions 107 can be printed at any position.

Optionally, at least one further print head 111-4 is used for applying the alkaline solution 108 to fix the coloring solutions 107, through which the alkaline solution 108 can be jet-printed onto one or more layers 117-1, . . . , 117-n. The print head 111-2 is also movable in two directions, so that the alkaline solution 108 can be printed at any position. The print heads 111-1, 111-2, 111-3 and 111-4 can be independently controllable or integrated in a common print module.

The production apparatus 200 provides a reduced number of possibly pre-colored and yttrium-doped neutral base slurries 109 in order to minimize the number of print heads 111-1 and 111-2 and still achieve an esthetic and functional result of the dental restoration 100. The final coloring is done separately by the selective application of coloring solutions 107.

The desired tooth color is composed and mixed from the different coloring solutions 107. A subtractive color system is used, which spans a limited dental color space (dental color gamut). The color mixture, the three-dimensional halftoning or dithering of these coloring solutions 107 in various ratios, is then created in the limited dental color space (gamut), which covers the common tooth colors, but not all colors.

In doing so, different coloring solutions 107 are selectively applied to a layer 117-1, . . . , 117-n using a 3D dithering algorithm, which is executed by a dithering module 113. For this purpose, the dithering module 113 comprises a processor for executing the 3D dithering algorithm and a digital data storage for storing the calculated mixing ratios and the dithering algorithm. The processor comprises any hardware system, component, or mechanism that processes data, signals, or other information. A processor may comprise a system with a central processing unit (CPU), multiple data processing units (MPU), dedicated electrical circuitry to achieve functionality, or other systems. The data storage may comprise hard disks, flash memory cards, random access memories (RAM) or read-only memories (ROM).

During dithering, the different coloring solutions are selectively applied in a certain ratio and two-dimensional printing pattern in the printing plane using the jet printing process. The 3D dithering algorithm also calculates that two-dimensional dithering patterns that are not the same are applied in several layers 117-1, . . . , 117-n on top of each other. This prevents optical artifacts such as stripes or moiré patterns on vertical surfaces. The color mixing, three-dimensional halftoning or dithering of these coloring solutions 107 in various ratios is created within the limited dental color space (gamut), which covers the common tooth colors, but not all general colors.

When using highly viscous slurries 109 with a viscosity of more than 100 mPas with a high degree of filling, special requirements are placed on the print head 111-1 and its fluid system. The print head 111-1 is compatible with the binder used and/or the carrier material of the slurry 109, such as water in the case of aqueous slurries, in order to avoid incompatibilities and consequences such as corrosion on the print head.

After the selective material application of a layer 117-1, . . . , 117-n of the slurry 109 by means of the jet printing process, this layer 117-1, . . . , 117-n is dried without cracking by evaporating the water or the solvent as a binder. The ceramic powder of the slurry 109 can already be colored in a color, for example a base color or tooth color. What remains is a porous white body layer having a layer thickness of 1 um to 50 um and a density of at least 2.5 g/cm³. This process is repeated until the entire dental restoration 100 is built up spatially in layers.

If two slurries 109 with different translucencies are available, the high-strength and opaque slurry 109 can be used for the dentin core and the normal-strength and highly translucent slurry 109 for the incisal edge. The selective coloring is carried out after 1−n layers 117-1, . . . , 117-n of the material application selectively jet printing coloring solutions, such as nitrate solutions (acids), according to a corresponding color coding or color information.

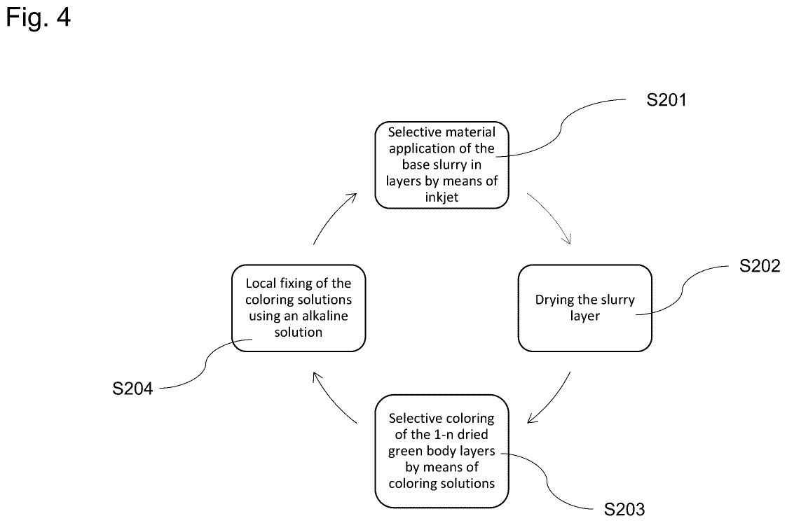

FIG. 4 shows a schematic view of a method of producing the dental restoration 100 by jet printing. In step S201, a selective material application of a slurry 109 in layers is first carried out by means of a jet printing process (inkjet process). In step S102, the applied slurry layer is dried, for example by supplying hot air. In step S203, the 1 to n applied layers 117-1, . . . , 117-n of the slurry 109 (green body layers) are selectively colored by means of coloring solutions. Here, the coloring solutions are also printed onto the 1 to n applied layers 117-1, . . . , 117-n by means of jet printing. In step S204, the printed coloring solutions are locally fixed by means of an alkaline solution 108 having a pH greater than 7. Subsequently, steps S201 to S204 are repeated until the dental restoration 100 is completely built up spatially.

For the method, either aqueous or solvent-based slurries 109 can be processed. Here, selective material application (step S201) and selective coloring (step S203) are carried out separately.

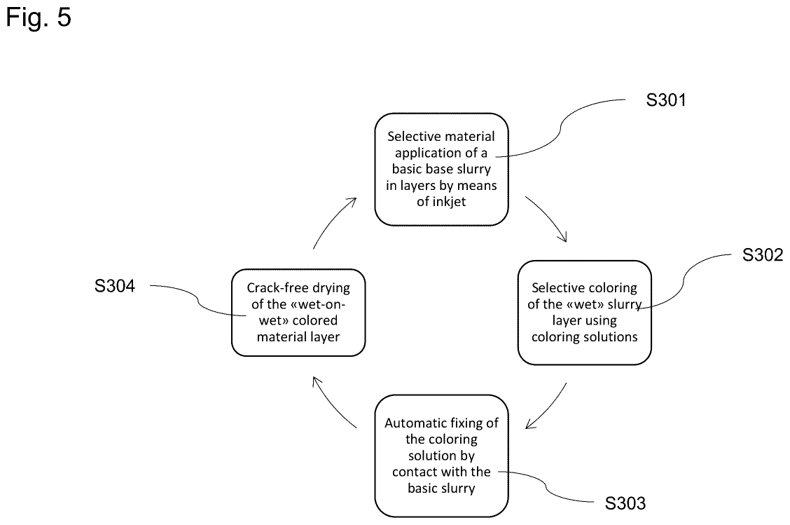

FIG. 5 shows a schematic view of a further method of producing the dental restoration 100 by jet printing. In step S301, a selective material application of a basic slurry 109 in layers is first carried out by means of a jet printing process (inkjet process).

The basic slurry 109 has a pH of greater than 7. A conventional basic slurry 109 contains H₂O, dispersing aids such as 0 to 5% by weight, preferably 0.01 to 5% by weight of carboxylic acid derivatives (citric acid) or 0 to 5% by weight of ammonium polyacrylic acid (NH4PAA), preferably 0.01 to 5% by weight. The pH (8-11) can be adjusted with NH4OH. The basic adjustment of the slurry 109 has the advantage that the ceramic slurry 109 behaves less aggressively in terms of corrosion towards the metal parts of the print head.

Furthermore, the slurry may contain small amounts, such as 0 to 5% by weight, preferably 0.001 to 5% by weight, of thickening agents to adjust the viscosity and/or the sedimentation behavior, such as polyvinyl pyrolidone, cellusol derivatives or xanthan gum. The sedimentation behavior ensures that the suspension is stable over a longer period of time and counteracts sedimentation.

In step S302, the still wet and undried applied layers 117-1, . . . , 117-n of the slurry 109 (green body layers) are selectively colored by means of the coloring solutions. This results in a precipitation reaction as soon as the coloring solution (nitric acid) comes into contact with the wet slurry 109. Dissolved ions of the 3d and 4f elements are precipitated as hydroxides due to the pH shift when they hit the wet layer. This results in the formation of Fe(OH)₃ or Er(OH)₃, for example.

In step S303, the coloring solutions are automatically fixed by contact with the basic slurry 109. In step S304, the wet-on-wet colored layers 117-1, . . . , 117-n are dried without cracking, for example by supplying hot air. Steps S301 to S304 are then repeated until the dental restoration 100 is completely built up spatially.

For the method, either aqueous or solvent-based slurries 109 can be processed. Selective material application (step S301) and selective coloring (step S302) are carried out separately.

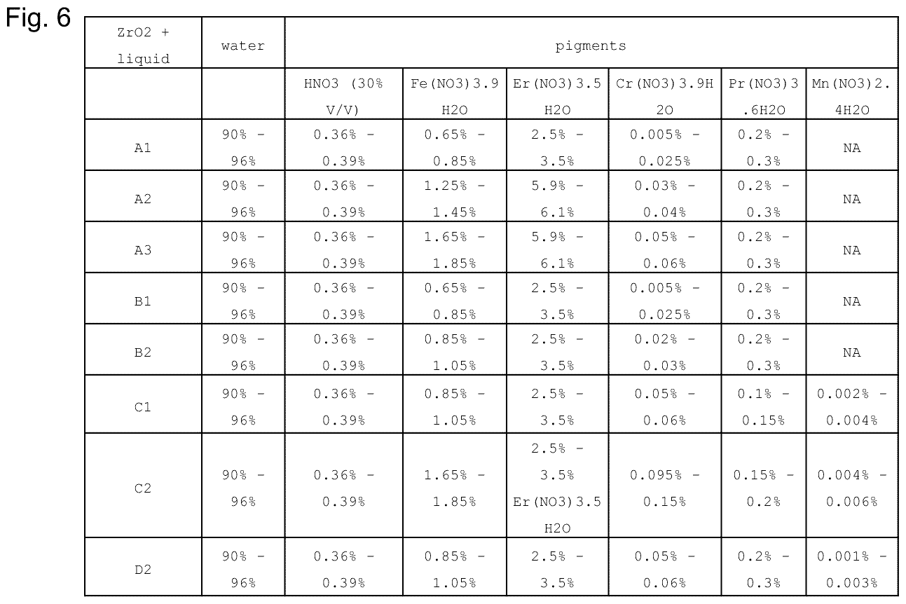

FIG. 6 shows a table with the compositions of different coloring solutions 107. The coloring solutions 107 are of low viscosity and can be jet-printed using either an inkjet print head with DOD technology or bubble jet technology. Several color channels can be integrated in a print head for multi-color printing. The print head can apply the coloring solution to the layer 117-1, . . . , 117-n at a high resolution of ≥720 dpi.

With six available print heads, only one print head is used for medium translucency slurries 109 and one print head for support material 110. With seven available print heads, two print heads are used for an opaque slurry 109 and a highly translucent slurry 109, respectively, and one print head is used for support material 110. The other four print heads are used for the coloring solutions 107.

The coloring solutions 107 are nitrate salt or chloride salt solutions on an aqueous basis. The coloring solution 107 comprises various metal salts (e. g. Fe(NO₃)₃·9H₂O; Pr(NO₃)₃·6H₂O; Tb(NO₃)₃·5H₂O; Er(NO₃)₃·5H₂O; Mn(NO₃)₂·4H₂O; Co(NO₃)₂·6H₂O; Cr(NO₃)₃·9H₂O; Mg(NO₃)₂·6H₂O; Al(NO₃)₃·9H₂O; Cu(NO₃)₂·3H₂O; Zn(NO₃)₂·6H₂O; Y(NO₃)₃·6H₂O; La(NO₃)₃·6H₂O; Ce(NO₃)₃·6H₂O; Nd(NO₃)₃·6H₂O; Sm(NO₃)₃·6H₂O; Gd(NO₃)₃·6H₂O; Yb(NO₃)₃·6H₂O; Ni(NO₃)₂·6H₂O; Co(NO₃)₂·6H₂O; Ga(NO₃)₃·xH₂O; In(NO₃)₃·xH₂O) dissolved in different concentrations. The concentration depends on the desired color intensity, but should not exceed the solubility limit of the salts. However, the coloring solutions 107 can also be significantly more concentrated depending on the desired composition. Increasing the ion concentration, however, increases the viscosity or shifts the pH into the acidic range (<7). A higher viscosity of the coloring solution is advantageous in order to reduce the penetration depth or diffusion depth of the coloring solution into the dried layers.

Water and nitric acid (HNO₃) can be used as the base acid. This means that only four nitrate salt solutions (Fe(NO₃)₃·9H₂O, Er(NO₃)₃·5H₂O, Cr(NO₃)₃·9H₂O, Mn(NO₃)₂·4H₂O, Tb(NO₃)₃·5H₂O and Pr(NO₃)₃·6H₂O) are used as the coloring solution 107 to produce the tooth color. At a minimum, only three nitrate salt solutions (Fe-yellow, Er-pink, Cr-gray) are required. The other salts are used for finer color adjustment.

In addition, the opacity of the slurry 109 of the dental restoration 100 can be adjusted using variable components of yttrium or ytterbium in the coloring liquid. For example, the elements yttrium, ytterbium, neodymium and europium are used as translucency enhancers and aluminum and silicon as opaque liquids. The yttrium content can be increased with a suitable coloring solution. In this way, different opacity values can be achieved in the dental restoration 100, such as opaque for the dentin core and translucent for the incisal edge.

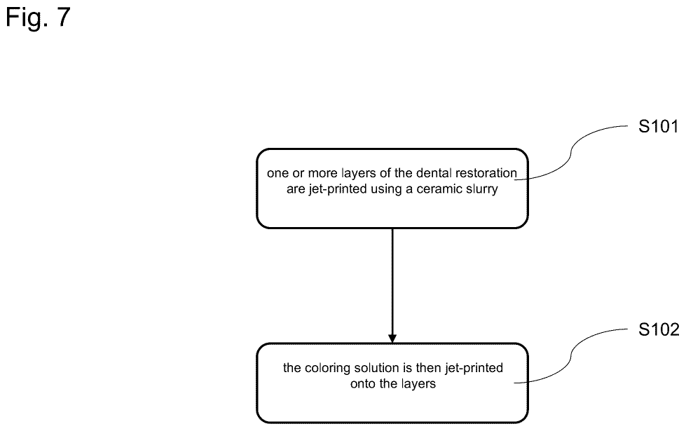

FIG. 7 shows a block diagram of the method of producing a dental restoration 100 by jet printing. In step S101, one or more layers 117-1, . . . , 117-n of the dental restoration 100 are jet-printed by means of the ceramic slurry 109. In step S102, the coloring solution 107 is then jet-printed onto the layers 117-1, . . . , 117-n.

If the infiltration depth of the coloring solution 107 is greater than a thickness of a single layer 117-1, . . . , 117-n, coloring can only take place after multiple layers of the slurry 109 have been applied. The penetration or diffusion depth of the coloring solution 107 can also be controlled by adjusting the viscosity. This is done by adding a suitable thickening agent that is stable in the pH range of the coloring solution. A suitable thickening agent is, for example, polyvinyl pyrolidone (PVP). The penetration or diffusion depth of all coloring solutions used can be brought to a uniform level in order to achieve the most stable process possible.

It makes sense to use different print head technologies, as the slurry 109 and the coloring solution 107 have different rheological properties. The slurry 109 is highly viscous (10-1000 mPas depending on the shear rate) and contains a high weight/volume proportion of an abrasive filler in the form of ceramic particles. A low viscosity of the slurry 109 between 10-500 mPas is advantageous and a viscosity between 10-100 mPas is even more advantageous. The higher the viscosity of the slurry 109, the more difficult it is to process using inkjet print heads based on MEMS technology.

The coloring solution 107, on the other hand, has a low viscosity (0.5-50 mPas). In addition, the volumes processed in each case are different and are in a volume ratio of approximately 98% for the slurry 109 and 2% for the coloring solution 107. The main volume processed is the slurry 109, whereas less volume of the coloring solution 107 is processed. The coloring solution 107 and the slurry 109 can be stored in differently sized receiving containers 119-1 and 119-2.

The coloring solution 107 can be jet-printed after the applied layers 117-1, . . . , 117-n have dried or on the wet layer 117-1, . . . , 117-n (wet-on-wet). When jet-printing the coloring solution 107 onto the dried layer 117-1, . . . , 117-n, the penetration depth of the coloring solution 107 should be controllable.

The fully built-up dental restoration 100 is then sintered in a sintering furnace. The slurries 109 in conjunction with the coloring solutions 107 can be adjusted to achieve uniform sintering behavior. The sintering kinetics can be homogeneously set by adjusting the coloring solution 107.

The sintering behavior of the individual layers can be adjusted by the targeted addition of sintering activators or sintering inhibitors to the coloring solution. Sintering activators are, for example, Zn²⁺, Al³⁺ or Mg²⁺ions that can be added to the coloring solution in the form of soluble salts, such as Zn(NO₃)₂·6H₂O, Al(NO₃)₃·9H₂O or Mg(NO₃)₂·6H₂O. Sintering inhibitors are e.g. La³⁺ or Y³⁺ which can be added to the coloring solution in the form of soluble salts, e.g. La(NO₃)₃·9H₂O or Y(NO₃)₃·6H₂O.

All the features explained and shown in connection with individual embodiments of the invention can be provided in different combinations in the subject-matter according to the invention in order to simultaneously realize their advantageous effects.

All method steps can be implemented by devices that are suitable for executing the respective method step. All functions performed by the features of the subject-matter can be a method step of a method.

The scope of protection of the present invention is given by the claims and is not limited by the features explained in the description or shown in the figures.

### REFERENCE LIST

100 Dental restoration
101 Dentin core
103 Tooth enamel
105 Tooth
107 Coloring solution
108 Alkaline solution
109 Slurry
110 Support material
111 Print head
113 Dithering module
115 Intermediate area
117 Layer
119 Receiving container

1. A method of producing a dental restoration by jet printing, comprising
jet printing one or more layers of the dental restoration using a ceramic slurry; and
jet printing a coloring solution onto the one or more layers.

2. The method according to claim 1,
wherein the one or more layers are dried before jet printing the coloring solution.

3. The method according to claim 1,
wherein the applied coloring solution is fixed by an alkaline solution.

4. The method according to claim 1,
wherein the ceramic slurry has a basic pH.

5. The method according to claim 4,
wherein the coloring solution is fixed by contact with the ceramic slurry.

6. The method according to claim 4,
wherein the one or more wet layers are dried together with the applied coloring solution.

7. The method according to claim 1,
wherein the steps for producing the dental restoration are repeated.

8. The method according to claim 1,
wherein the produced dental restoration is subjected to a drying and/or debinding step prior to a sintering process.

9. The method according to claim 1,
wherein the dental restoration is sintered in a sintering furnace.

10. The method according to claim 1,
wherein jet printing the coloring solution onto the one or more layers comprises using different two-dimensional dithering patterns in successive layers of the dental restoration.

11. A production apparatus for producing a dental restoration by jet printing, comprising:
a first print head for jet printing one or more layers of the dental restoration using a ceramic slurry; and
a second print head for jet printing a coloring solution onto the one or more layers.

12. The production apparatus according to claim 11, wherein the production apparatus comprises a receiving container for the slurry and a receiving container for the coloring solution.

13. The production apparatus according to claim 11,
wherein the first print head and the second print head are integrated in a common print module.

14. The production apparatus according to claim 11,
wherein the first print head and the second print head are independently controllable.

15. The production apparatus according to claim 11,
wherein the production apparatus comprises a dithering module for calculating intermediate color values by mixing at least two coloring solutions.

16. The production apparatus according to claim 14,
wherein the dithering module is configured to use different two-dimensional dithering patterns in successive layers of the dental restoration.

17. A jet-printing system, comprising:
a production apparatus for producing a dental restoration by jet-printing;
at least one ceramic slurry for jet printing one or more layers of the dental restoration; and
a coloring solution for jet printing onto the one or more layers.

18. The jet-printing system according to claim 17,
wherein the at least one ceramic slurry and/or coloring solution is stored in a container.

19. The jet-printing system according to claim 18,
wherein the container is replaceable.
